(19) United States
(12) Patent Application Publication
(10) Pub. No.: US 2016/0051496 A1
(43) Pub. Date: Feb. 25, 2016

(54) Oral Transmucosal Compositions Including C-SERMs for Treating Female Infertility

(71) Applicants: Tsu-I Catherine Wang, Sugar Land, TX (US); Bruce Vincent Biundo, Houston, TX (US)

(72) Inventors: Tsu-I Catherine Wang, Sugar Land, TX (US); Bruce Vincent Biundo, Houston, TX (US)

(21) Appl. No.: 14/799,291

(22) Filed: Jul. 14, 2015

Related U.S. Application Data
(63) Continuation-in-part of application No. 14/591,748, filed on Jan. 7, 2015.
(60) Provisional application No. 62/039,810, filed on Aug. 20, 2014.

(51) Int. Cl. A61K 31/138; A61K 9/00

(52) U.S. Cl. CPC A61K 31/138; A61K 9/006

(57) ABSTRACT

Formulations for oral transmucosal compositions including clomiphene-like selective estrogen receptor modulators (C-SERMs) in combination with transmucosal absorption enhancers are disclosed. Oral transmucosal compositions can be for fast release or slow release, and can be administered to induce ovulation in a female patient and thereby reduce symptoms of anovulatory infertility, unexplained infertility, and the like. Oral transmucosal compositions include liquid dosage forms, solid dosage forms, and chewing gums. Further dosage forms include mucoadhesive thin strips, thin films, tablets, patches, and tapes, among others. Other dosage forms are: mucoadhesive liquids, such as, for example gel-forming liquid; gel-forming semisolids; and gel-forming powders, among other dosage forms that exhibit mucoadhesive properties, and provide oral transmucosal delivery of C-SERMs. Oral transmucosal compositions will deliver C-SERMs directly into the patient's bloodstream, and provide high bioavailability of C-SERMs; therefore, the required doses are lower.

### CROSS-REFERENCE TO RELATED APPLICATIONS

This application claims the benefit of U.S. application Ser. No. 14/591,748, filed Jan. 7, 2015, which claims priority to U.S. Provisional Application Ser. No. 62/039,810, filed Aug. 20, 2014, which are hereby incorporated by reference.

### BACKGROUND

1. Field of the Disclosure

The present disclosure relates generally to pharmaceutical compositions, and more particularly, to oral transmucosal compositions including clomiphene-like selective estrogen receptor modulators (C-SERMs) for treating female infertility.

2. Background Information

Infertility can be defined as the inability to achieve pregnancy in a one-year period of regular unprotected sexual intercourse. Female infertility can refer to the inability to conceive and/or to carry a pregnancy to term. Despite the difficulties in estimating the prevalence of infertility, it is generally accepted that one out of every four women is infertile during one or more periods of time throughout the reproductive portion of her lifetime.

The main causes of infertility among women include ovulatory disorders, reproductive tract pathologies, reduced oocyte quality and follicular depletion inherent to aging. Some forms of ovulatory disorders include when ovulation does not occur because of the inability of the hypothalamus to secrete gonadotropin-releasing hormone (GnRH), which stimulates the pituitary gland to produce the luteinizing hormone (LH) and follicle-stimulating hormone (FSH) that triggers ovulation. Ovulation problems may also be due to polycystic ovary syndrome (PCOS), thyroid gland disorders, adrenal gland disorders, excessive exercise, diabetes, weight loss, obesity, or psychological stress. There are also a considerable number of women with unexplained infertility, also called idiopathic infertility. One of the possible causes of idiopathic infertility could be related to deficiencies in the implantation process.

Conventional treatments for female infertility include the administration of active pharmaceutical ingredients (APIs) for treating infertility related to hormonal problems (e.g., ovulation disorders), in vitro fertilization, and intrauterine insemination techniques. All these treatments involve APIs being administered through various oral and injectable delivery methods.

Clomiphene is a selective estrogen receptor modulator that has been used for treating ovulation disorders. Selective Estrogen Receptor Modulators (SERMs) are structurally unique compounds that interact with intracellular estrogen receptors in target organs. SERMs can possess either antagonist or agonist properties, and in certain cases, may possess both properties. Some SERMs, such as tamoxifen and raloxifene possess estrogen agonist properties that cause unusual pharmacological effects to be exhibited when these particular SERMs interact with certain tissues (e.g., bone, liver and cardiovascular system tissues). Additionally, these same SERMs possess estrogen antagonist properties when these particular SERMs interact with other tissues (e.g., brain and breast tissues). Finally, these same SERMs possess mixed agonist/antagonist properties when interacting with uterine tissue. Clomiphene and SERMs that mimic clomiphene act specifically as an estrogen antagonist in the brain, specifically in the hypothalamus and pituitary sites.

Within the estrogen-negative feedback mechanism, estradiol slows down the release of GnRH from the hypothalamus which in turn results in the reduction of LH/FSH production by the pituitary gland. Clomiphene acts to increase the production and release of GnRH by the hypothalamus as well as production and release of LH and FSH from the pituitary gland. LH and FSH then act on the ovaries to increase ovarian follicular activity and eventual ovulation.

Oral dosage forms usually subject the API to degradation in the gastrointestinal tract and the first pass metabolism in the liver, and are commonly associated with a delayed onset of the effects of the treatment. Injections and implanted pellets can cause local pain to the recipient as well as require the assistance of health care professionals thereby making these dosage delivery forms inconvenient and expensive.

Transdermal administration (e.g., implanted pellets, patches, gels, etc.) possesses the benefits of the avoidance of the first-pass metabolism as well as degradation in the gastrointestinal tract. Transdermal administration typically includes the added benefit that the treatment is not painful. Unfortunately, transdermal compositions, excluding implant pellets, are often associated with low percentages of absorption through the skin. Another drawback is that a large part of the API remains on the skin with the potential risk of being transferred to another person through direct skin-to-skin contact. Additionally, the non-absorbed portion of the APIs is lost to the surrounding environment making these formulations non-environmentally-friendly.

Oral transmucosal delivery is a particularly advantageous delivery route because it is a non-invasive drug delivery method. Oral transmucosal delivery promotes better patient compliance and involves lower costs than invasive procedures, such as, injection and implantation of pellets. Oral transmucosal delivery also results in a much shorter onset time (e.g., the time from administration to therapeutic effect) than oral delivery and may be easily self-administered. Oral transmucosal administration involves the patient holding the composition within the oral cavity (e.g., between the cheek and gum, beneath the tongue, etc.) while the API dissolves in the available fluid (e.g., saliva), diffuses through the mucosa lining of the mouth, and enters the bloodstream bypassing the gastrointestinal tract as well as hepatic metabolism.

### SUMMARY

The present disclosure refers to oral transmucosal compositions that include one or more clomiphene-like selective estrogen receptor modulators (C-SERMs) as APIs in combination with transmucosal absorption enhancers to induce ovulation in a female patient and reduce symptoms of anovulatory infertility, unexplained infertility, and the like. In some embodiments, APIs include C-SERMs, such as, clomiphene (Clomid®), analogs thereof, or any other chemical compound that acts on estrogen receptors to block the normal estrogen feedback control of the hypothalamus and subsequent negative feedback control on the pituitary gland.

In some embodiments, the C-SERM employed in oral transmucosal compositions is clomiphene. In these embodiments, clomiphene within oral transmucosal compositions is implemented as clomiphene citrate or an analog thereof. In other embodiments, clomiphene implemented within oral transmucosal compositions is zuclomiphene, enclomiphene, or a combination of these two clomiphene isomers.

In some embodiments, transmucosal absorption enhancers provide more efficient penetration of API through oral mucosal tissue. In these embodiments, the transmucosal absorption enhancers allow lower API dosage requirements.

In some embodiments, the amount of absorption enhancers included in oral transmucosal compositions range from about 0.1% to about 20%; with the most suitable amount being about 1% to about 10%. These percent ranges may refer to % weight by weight, % weight by volume, or % volume by volume.

In some embodiments, oral transmucosal compositions allow the delivery of C-SERMs directly into the patient's bloodstream bypassing the gastrointestinal tract and the hepatic metabolism. Bypassing the hepatic metabolism results in a higher percentage of bioavailability of C-SERMs to the patient.

In some embodiments, oral transmucosal compositions include different components, such as, APIs, transmucosal absorption enhancers, suitable vehicles, and suitable additives, among others. In these embodiments, various additives are included to facilitate the preparation of suitable dosage forms. For example, additives include solvents, diluents, binders, disintegrants, lubricants, glidants, mucoadhesive polymers, thickening agents, transmucosal absorption enhancers, polymer plasticizers, pH adjusters, preservatives, sweeteners, flavors, colors, effervescent agents, stabilizing agents, antioxidants, and surfactants, among others.

In some embodiments, oral transmucosal compositions include liquid dosage forms, such as, for example sublingual solutions, emulsions, suspensions, and liquid sprays, among others. In other embodiments, oral transmucosal compositions include solid dosage forms, such as, sublingual tablets and buccal troches, among others. In yet other embodiments, oral transmucosal dosage forms include chewing gums.

In some embodiments, oral transmucosal dosage forms include mucoadhesive polymers as part of the compositions. Examples of dosage forms having mucoadhesive polymers include mucoadhesive thin strips, thin films, tablets, patches, and tapes, among others. In other embodiments, dosage forms include: mucoadhesive liquids, such as, gel-forming liquids; semisolids, such as, for example gels, gel-forming ointments, and gel-forming pastes; gel-forming powders; or any other dosage forms that exhibit mucoadhesive properties and provide oral transmucosal delivery of C-SERMs.

In some embodiments, oral transmucosal compositions are administered in the oral cavity at the sublingual, palatal, buccal, gingival, or the like.

In some embodiments, oral transmucosal compositions can be tailored for individual patients according to clinical symptoms and baseline serum concentrations of estradiol, LH, and/or progesterone. These oral transmucosal compositions can be prescribed with various concentrations of C-SERMs, and suitable dosage regimens to more closely mimic the circadian rhythm and physiological pulsatile secretion of GnRH, thereby keeping the LH/FSH and estradiol levels within physiologic ranges suitable for inducing conception.

In some embodiments, oral transmucosal compositions are administered within a dosage range from about 5 mg/day to about 100 mg/day of clomiphene, preferably from about 25 mg/day to about 50 mg/day. In these embodiments, oral transmucosal compositions are administered for about 5 days and starting at or around days 2-5 of the menstrual cycle, at the convenience of the amenorrheic or oligomenorrheic patient, or at the recommendation of the treating physician.

In some embodiments, oral transmucosal dosage forms are designed for fast release and transmucosal absorption of C-SERMs. In other embodiments, oral transmucosal dosage forms are designed for slow release and absorption of C-SERMs over a prolonged period of time.

In some embodiments, a low dose C-SERM in any of the above identified dosage forms can result in acceptable ovulation inducting levels in the patient.

Numerous other aspects, features, and benefits of the present disclosure may be made apparent from the following detailed description.

### DETAILED DESCRIPTION

The present disclosure is described here in detail. Other embodiments may be used and/or other changes may be made without departing from the spirit or scope of the present disclosure. The described embodiments are not meant to limit the subject matter presented here.

### DEFINITIONS

As used here, the following terms have the following definitions:

“Absorption Enhancer” or, equivalently, “Penetration Enhancer” refers to a substance used to increase the rate of permeation through the mucous membrane, skin or other body tissue of one or more substances (e.g., APIs) in a formulation.

“Active Pharmaceutical Ingredients (APIs)” refer to chemical compounds that induce a desired effect, and include agents that are therapeutically or prophylactically effective.

“Anovulatory Infertility” refers to a condition in which there is no rupture of the follicle with subsequent release of an ovum.

“Clomiphene-like SERMs (C-SERMs)” refer to chemical compounds that act like clomiphene, as selective estrogen antagonist in the brain, specifically in the hypothalamus and pituitary sites. As such, C-SERMs act to increase GnRH secretion to stimulate increased pituitary gonadotropin release that, in turn, drives ovarian follicular activity.

“Selective Estrogen Receptor Modulators (SERMs)” refer to chemical compounds that interact with intracellular estrogen receptors in target organs.

“Treating” and “Treatment” refers to reduction in severity and/or frequency of symptoms, elimination of symptoms and/or underlying cause, prevention of the occurrence of symptoms and/or their underlying cause, and improvement or remediation of damage.

“Vehicle” refers to a substance of no therapeutic value that is used to convey at least one API for administration.

### DESCRIPTION OF THE DISCLOSURE

Embodiments of the present disclosure are directed towards oral transmucosal delivery of active pharmaceutical ingredient (APIs). Oral transmucosal compositions that include clomiphene-like selective estrogen receptor modulators (C-SERMs) as APIs in combination with transmucosal absorption enhancers are disclosed. These oral transmucosal compositions are proposed to induce ovulation in a female patient and reduce symptoms of anovulatory infertility, unexplained infertility, and the like.

As described previously, estradiol serves as a major mediator of sex steroid-gonadotropin feedback. Thus, the secretion of luteinizing hormone (LH) and follicle-stimulating hormone (FSH) are, to a large extent, modified by C-SERMs that affect the activity of estradiol. C-SERMs possess the capacity to blunt the activity of estradiol by competing with estradiol for the estrogen receptors of the hypothalamus and pituitary gland thereby increasing the amount of LH and FSH the body produces. These increased levels of LH and FSH correspond with increased ovarian follicular activity and eventual ovulation. Therefore, C-SERMs can be used for anovulatory infertility (e.g., anovulation and oligoovulation), unexplained infertility, and the like.

Formulation

In some embodiments, oral transmucosal compositions include one or more C-SERMs as APIs, transmucosal absorption enhancers, vehicles, and additives, among other suitable ingredients. In these embodiments, APIs include C-SERMs, such as, clomiphene (Clomid®), analogs thereof, or any other chemical compound that acts on estrogen receptors to block the normal estrogen feedback control of the hypothalamus and subsequent negative feedback control on the pituitary gland.

In some embodiments, the C-SERM employed in oral transmucosal compositions is clomiphene. In these embodiments, clomiphene within oral transmucosal compositions is implemented as clomiphene citrate or an analog thereof. In other embodiments, clomiphene implemented within oral transmucosal compositions is zuclomiphene, enclomiphene, or a combination of these two clomiphene isomers. The list of C-SERMs above is not exhaustive; other compounds described in the art that meet the set requirements can also be considered.

In some embodiments, various additives are included to facilitate the preparation of suitable dosage forms. For example, additives include solvents, diluents, binders, disintegrants, lubricants, glidants, mucoadhesive polymers, thickening agents, transmucosal absorption enhancers, polymer plasticizers, pH adjusters, preservatives, sweeteners, flavors, colors, effervescent agents, stabilizing agents, antioxidants, and surfactants, among others.

In some embodiments, diluents for solid dosage forms include calcium carbonate, dibasic calcium phosphate, tribasic calcium phosphate, calcium sulfate, kaolin, microcrystalline cellulose, and other cellulose derivates, sodium chloride, starch and starch derivates, sucrose, dextrose, lactose, and sorbitol, among others.

Binders for solid dosage forms include starch and starch derivatives, gelatin, sucrose, glucose, dextrose, molasses, lactose, natural and synthetic gums, acacia, sodium alginate, extract of Irish Moss, panwar gum, ghatti gum, mucilage of isapol husks, carboxymethylcellulose, methylcellulose, cellulose derivatives, veegum, polyvinylpyrolidone, and polyethylene glycols, among others.

Disintegrants for solid dosage forms include veegum, agar, bentonite, alginic acid and alginic acid derivatives, guar gum, starch, sodium starch glycolate, other starch derivatives, clays, cellulose, and cellulose derivatives, among others.

Lubricants for solid dosage forms include stearic acid, stearic acid derivatives, stearic acid salts such as magnesium stearate and calcium stearate, talc, hydrogenated vegetables oils, polyethylene glycols, surfactants, and waxes, among others.

Additionally, solid dosage forms of oral transmucosal compositions include: a glidant, such as, colloidal silicon dioxide and talc, among others; a sweetening agent, such as, sucrose or saccharin, among others; natural or artificial flavors, such as, for example peppermint, methyl salicylate, or orange flavor, among others.

The pH adjusting agents include sodium bicarbonate, magnesium hydroxide, calcium carbonate, dibasic calcium phosphate, tribasic calcium phosphate, sodium bicarbonate, magnesium hydroxide, potassium hydroxide, citric acid, lactic acid, hydrochloric acid, sulfuric acid, phosphoric acid, sodium phosphate monobasic, and sodium phosphate dibasic, among others.

Surfactants include: polysorbates, such as, polysorbate 20, 40, 60, and 80, among others; sorbitan esters, such as, for example sorbitan monolaurate, sorbitan monopalmitate, sorbitan monooleate, among others; and sodium lauryl sulfate, among others.

Effervescent agents are usually a combination of one or more acids with one or more bases. Acids are selected from citric acid, tartaric acid, and the like. Bases can be sodium bicarbonate or other suitable agents that may react with acids, and produce gas.

In some embodiments, a stabilizing agent is used to stabilize the API for a specific dosage form. In these embodiments, the stabilizing agent used will depend on the API used as well as the other additive ingredients. Any suitable chemical substance may be used as a stabilizing agent. Stabilizing agents are known to those skilled in the art and therefore will not be discussed further herein.

Mucoadhesive polymers include: gums, such as, for example acacia, agarose, alginic acid, sodium alginate and other alginic acid derivatives, carrageenan, gelatin, gellan, guar gum, hakea gum, karaya gum, and locust bean gum, among others; chitosan and chitosan derivatives; hyaluronic acid, pectin, and other polysaccharides; gelatin, polyisoprene, polyisobutylene, polyetherurethane, polyvinylalcohol, polyvinylpyrrolidone, polycarbophil, polyethylene oxide polymers, and pullulan, among others. Mucoadhesive polymers also include cellulose derivatives such as ethyl cellulose, cellulose acetate, hydroxyethyl cellulose, hydroxypropylcellulose, hydroxypropylmethylcellulose, methylcellulose, methylhydroxyethylcellulose, and sodium carboxymethyl cellulose, among others; poly(acrylic acid)-based polymers such as polyacrylates, poly (methylvinylether-co-methacrylic acid), poly(acrylic acid-co-ethylhexylacrylate), poly(acrylic acid-co-acrylamide), poly(acrylic acid-co-butylacrylate), poly(acrylic acid-co-methyl methacrylate), poly (2-hydroxyethyl methacrylate), polymethacrylates, poly(alkylcyanoacrylate) and other cyanoacrylates, poly (isohexycyanoacrylate), poly (isobutylcyanoacrylate), and hydroxyethyl methacrylate, and any other polymer known to a person skilled in the art that exhibits mucoadhesive characters.

Plasticizers for mucoadhesive polymeric dosage forms include pullulan, hydroxypropyl methylcellulose, propylene glycol, glycerol, sorbitol, mannitol, polyethylene glycols (PEG 200, 400, 600, 1000, 1500, 2000), tartaric acid, malic acid, lactic acid, citric acid, and yonkenafil, and any other chemical known to a person skilled in the art that can increase the plasticity of any mucoadhesive polymer.

Bases for chewing gum include cellulosic polymer, and acrylic polymer, among others.

In some embodiments, transmucosal absorption enhancers provide more efficient penetration of API through oral mucosal tissue. In these embodiments, the transmucosal absorption enhancers allow lower API dosage requirements.

Oral transmucosal absorption enhancers include: enzyme inhibitors, such as, aprotinin and puromycin, among others; chitosan and chitosan derivatives such as chitosan glutamate, trimethyl chitosan, chitosan-4-thioglycolic acid, 5-methyl-pyrrolidine chitosan, and chitosan-4-thio-butylamidine, among others; alpha, beta, and gama cyclodextrins, such as, for example dimethyl cyclodextrin, sulfobutyl cyclodextrin, 2-hydroxypropyl-beta-cyclodextrin, poly-beta-cyclodextin, and methylated beta-cyclodextrin, among others; bile salts, such as, for example sodium deoxycholate, sodium glycocholate, sodium glycodeoxycholate, sodium glycodihydrofusidate, sodium taurocholate, sodium taurodeoxycholate, sodium tauroglycocholate, sodium taurodihydrofusidate, and sodium ursocholate, among others; chelating agents, such as, for example sodium EDTA, citric acid, sodium citrate, sodium salicylate, methylsalicylate, methoxysalicylate, and polyacrylates, among others; alcohols, such as, ethanol and isopropanol, among others; fatty acids and derivatives, such as, for example oleic acid, methyloleate, capric acid, neodecanoic acid, elaidic acid, lauric acid, palmitoylearnitine, cod liver oil extract, mono glycerides and diglycerides of oleic acid and capric acid, lauric acid, sodium laurate, linoleic acid, sodium fusidate, sodium caprate, lyceryl monolaurate, glyceryl monooleate, glyceryl monostearate, sucrose fatty acid esters, and diethylene glycol monoethyl ether, among others; lecithins and phospholipids, such as, for example phodphatidylcholine, lysophosphatidyl choline, and didecanoylphophatidylcholine, among others; sulfoxides, such as, dimethylsulfoxide and decylmethyl sulfoxide, among others; polyols, such as, for example glycerin, propylene glycol, propanediol, and polyethylene glycols of various molecular weights, among others; urea and derivatives, such as, unsaturated cyclic urea, among others; surfactants, such as, for example sodium dodecyl sulfate, sodium lauryl sulfate, dioctyl sodium sulfosuccinate, nonylphenoxypolyoxyethylene, polyoxyethylene alkyl ethers, polyoxyethylene-9-lauryl ether, polyoxyethylene 23 lauryl ether, polyoxyethylene-20-cetyl ether, polyethyleneglycol dodecyl ether, polyethylene glycol-8 laurate, glyceryl monolaurate, polyoxyethylene stearates, polysorbates, sorbitan fatty acid esters, polyoxyethylene castor oil derivatives, benzalkonium chloride, cetylpyridinium chloride, and cetyltrimethylammonium bromide, among others. Other oral transmucosal absorption enhancers include alkylglycosides, azone, hyaluronic acid, sodium Hyaluronate, glycine chenodeoxycholate, lauroyl macroglycerides, isopropyl myristate, isopropyl palmitate, glutathione, witepsol, menthol, capsaicin, taurine, tocopheryl acetate, lauroyl macroglycerides, lionoleoyl polyoxyl-6 glycerides; diethylene glycol monoethyl ether, dextran sulfate, various saponins, poly-1-arginine, and 1-lysine, and any other chemical known to a person skilled in the art that exhibits penetration enhancing effect on transmucosal absorption.

In some embodiments, the amount of absorption enhancers included in oral transmucosal compositions range from about 0.1% to about 20%; with the most suitable amount being about 1% to about 10%. These percent ranges may refer to % weight by weight, % weight by volume, or % volume by volume.

In some embodiments, oral transmucosal compositions include pharmaceutical solvents to produce sprays, solutions, emulsions, suspensions, gels, gel-forming liquids, ointments and pastes, among others. In these embodiments, pharmaceutical solvents for liquid dosage forms of oral transmucosal compositions include water, glycerin, propylene glycol, liquid polyethylene glycols of various molecular weights, ethyl oleate, medium chain triglycerides, isopropyl myristate, isopropyl palmitate, isopropyl stearate, other pharmaceutically acceptable esters of C8-C22 fatty acids and C2-C6 alcohols, mineral oil, and vegetable oils, among others. Further to these embodiments, C8-C22 fatty acids include fatty acids having from 8 to 22 carbon atoms, such as, for example myristic acid, palmitic acid, stearic acid, arachidic acid, or oleic acid, among others. Still further to these embodiments, C2-C6 alcohols include alcohols having from 2 to 6 carbon atoms, in particular the C2-C5 alcohols as well as the homologues with 6 carbon atoms including diols and triols, such as, for example ethanol, propylene glycol, and glycerol, among others. Examples of vegetable oils include almond oil, peanut oil, sesame oil, sunflower oil, safflower oil, canola oil, corn oil, and olive oil, among others.

In some embodiments, oral transmucosal ointments and pastes include petrolatum, PCCA Plasticized™ base, paraffin wax, various synthetic wax, lanolin, beeswax, carnauba wax, candelila wax, silicones, isopropylesters, polyols, cellulose ethers, among other suitable bases. In addition, ointment bases also include suitable pharmaceutical solvents, such as water, liquid polyethylene glycols of various molecular weights, ethyl oleate, medium chain triglycerides, isopropyl myristate, isopropyl palmitate, isopropyl stearate, and other pharmaceutically acceptable esters of C8-C22 fatty acids and C2-C6 alcohols, mineral oil, and vegetable oils, among others.

Administration

In some embodiments, oral transmucosal compositions allow the delivery of C-SERMs directly into the patient's bloodstream bypassing the gastrointestinal tract and the hepatic metabolism. Bypassing the hepatic metabolism results in a higher percentage of bioavailability of C-SERMs to the patient.

In some embodiments, oral transmucosal compositions are administered in the oral cavity at the sublingual, palatal, buccal, gingival, or the like. Oral transmucosal compositions may be self-administered by the patient or administered by a medical practitioner, such as a physician or nurse.

In some embodiments, oral transmucosal compositions include liquid dosage forms, such as, for example sublingual solutions, emulsions, suspensions, and liquid sprays, among others. In other embodiments, oral transmucosal compositions include solid dosage forms, such as, sublingual tablets and buccal troches, among others. In yet other embodiments, oral transmucosal dosage forms include chewing gums.

In some embodiments, oral transmucosal dosage forms include mucoadhesive polymers as part of the compositions. Examples of dosage forms having mucoadhesive polymers include mucoadhesive thin strips, thin films, tablets, patches, and tapes, among others. In other embodiments, dosage forms include: mucoadhesive liquids, such as, gel-forming liquids; semisolids, such as, for example gels, gel-forming ointments, and gel-forming pastes; gel-forming powders; or any other dosage forms that exhibit mucoadhesive properties and provide oral transmucosal delivery of C-SERMs.

In some embodiments, oral transmucosal dosage forms are designed for fast release and transmucosal absorption of C-SERMs. In other embodiments, oral transmucosal dosage forms are designed for slow release and absorption of C-SERMs over a prolonged period of time.

In some embodiments, oral transmucosal compositions are administered in a single administration whereby a certain amount of C-SERM is administered together. In an example, one puff of a spray solution is administered representing the full desired dose. In other embodiments, oral transmucosal compositions are administered by multiple administrations in one or more sub-doses over a specified period of time. In an example, one, two or more puffs of a smaller dose of the oral transmucosal composition are administered—preferably one after another in quick succession.

In some embodiments, oral transmucosal compositions can be tailored for individual patients according to clinical symptoms and baseline serum concentrations of estradiol, LH, and/or progesterone. These oral transmucosal compositions can be prescribed with various concentrations of C-SERMs, and suitable dosage regimens to more closely mimic the circadian rhythm and physiological pulsatile secretion of GnRH, thereby keeping the LH/FSH and estradiol levels within physiologic ranges suitable for inducing conception.

In some embodiments, the dosages (e.g., daily) required depend on the type of C-SERM included in the disclosed oral transmucosal compositions. In other words, some C-SERMs are more potent than others, and hence, the dosage regimen varies among the various C-SERMs used. In these embodiments, a low dose C-SERM in any of the above identified dosage forms can result in acceptable ovulation inducting levels in the patient.

In some embodiments, oral transmucosal compositions are administered within a dosage range from about 5 mg/day to about 100 mg/day of clomiphene, preferably from about 25 mg/day to about 50 mg/day. In these embodiments, oral transmucosal compositions are administered for about 5 days and starting at or around days 2-5 of the menstrual cycle, at the convenience of the amenorrheic or oligomenorrheic patient, or at the recommendation of the treating physician.

The following examples are intended to illustrate the scope of the disclosure and are not intended to be limiting. It is to be understood that other pharmaceutical formulations known to those skilled in the art may alternatively be used.

### Examples

Exemplary dosage forms of the oral transmucosal compositions are described below.

Example #1 illustrates formula for one clomiphene citrate sublingual tablet:

| Ingredient | Composition |
|---|---|
| Clomiphene citrate | 25 mg |
| Penetration enhancer(s) | 1-10% |
| Flavor(s) | 0.5-5% |
| Lactose/sucrose (80:20) | q.s. 150-200 mg |

Example #2 illustrates formula for one dose of clomiphene citrate sublingual drops:

| Ingredient | Composition |
|---|---|
| Clomiphene citrate | 25 mg |
| Co-solvent(s) | 10-50% |
| Penetration enhancer(s) | 1-10% |
| Flavor(s) | 0.5-5% |
| Sweetener(s) | 0.1-1.5% |
| Base Solvent | q.s. 0.2 mL |

Example #3 illustrates formula for one dose of clomiphene citrate oral adhesive paste:

| Ingredient | Composition |
|---|---|
| Clomiphene citrate | 25 mg |
| Gelatin | 1-5% |
| Pectin | 1-5% |
| Sodium | 1-10% |
| Carboxymethylcellulose | |
| Xanthan gum | 0.1-5% |
| PEG-90M | 1-10% |
| Penetration enhancer(s) | 1-10% |
| Flavor(s) | 0.5-5% |
| Sweetener(s) | 0.1-1.5% |
| PCCA Plasticized ™ Base* | q.s. 0.2-0.5 mL |

*It is a proprietary gel base produced by Professional Compounding Centers of America (PCCA)

While various aspects and embodiments have been disclosed, other aspects and embodiments are contemplated. The various aspects and embodiments disclosed are for purposes of illustration and are not intended to be limiting, with the true scope and spirit being indicated by the following claims.

1. A pharmaceutical composition for inducing ovulation comprising one or more clomiphene-like selective estrogen receptor modulators (C-SERMs), wherein the dosage form of the composition is oral transmucosal enabling delivery of C-SERMs directly into a patient's bloodstream.

2. The pharmaceutical composition of claim 1, wherein the C-SERM is clomiphene or analogs thereof.

3. The pharmaceutical composition of claim 2, wherein the clomiphene is clomiphene citrate or an analog thereof.

4. The pharmaceutical composition of claim 1, wherein the C-SERM is enclomiphene, zuclomiphene, or combinations thereof.

5. The pharmaceutical composition of claim 1, wherein clomiphene is administered at about 5 mg/day to about 100 mg/day.

6. The pharmaceutical composition of claim 1, wherein the oral transmucosal administration is sublingual, palatal, buccal, or gingival.

7. The pharmaceutical composition of claim 1, wherein the dosage form of the composition is selected from the group consisting of: a solid, a liquid, a semi-solid, a chewing gum, a gel-forming liquid, and gel-forming powder.

8. The pharmaceutical composition of claim 7, wherein the solid dosage form is a sublingual tablet or a buccal troche.

9. The pharmaceutical composition of claim 7, wherein the liquid dosage form is selected from the group consisting of: sublingual solution, emulsion, suspension, and liquid spray.

10. The pharmaceutical composition of claim 7, wherein the semi-solid dosage form is selected from the group consisting of: gel, gel-forming ointment, and gel-forming paste.

11. The pharmaceutical composition of claim 1, further comprising one or more additives selected from the group consisting of: solvents, diluents, binders, disintegrants, lubricants, glidants, mucoadhesive polymers, thickening agents, penetration enhancers, polymer plasticizers, pH adjusters, preservatives, sweeteners, flavors, colors, effervescent agents, stabilizing agents, antioxidants, and surfactants.

12. The pharmaceutical composition of claim 11, wherein at least one solvent is present at about 10% to 50% of the composition.

13. The pharmaceutical composition of claim 11, wherein the diluents selected are lactose and sucrose that are present in an 80:20 ratio.

14. The pharmaceutical composition of claim 11, wherein the penetration enhancer is an oral transmucosal absorption enhancer.

15. The pharmaceutical composition of claim 14, wherein the oral transmucosal absorption enhancer is selected from the group consisting of: enzyme inhibitors; chitosan or chitosan derivative; cyclodextrins; bile salts; chelating agents; alcohols; fatty acids and derivatives thereof; lecithins; sulfoxides; polyols; urea and derivatives thereof; surfactants; alkylglycosides, azone, hyaluronic acid, sodium hyaluronate, glycine chenodeoxycholate, lauroyl macroglycerides, isopropyl myristate, isopropyl palmitate, glutathione, witepsol, menthol, capsaicin, taurine, tocopheryl acetate, lauroyl macroglycerides, lionoleoyl polyoxyl-6 glycerides; diethylene glycol monoethyl ether, dextran sulfate, saponins, poly-I-arginine, and I-lysine.

16. The pharmaceutical composition of claim 14, wherein the penetration enhancer is present at about 0.1% to about 20% of the composition.

17. The pharmaceutical composition of claim 14, wherein clomiphene is clomiphene citrate or analogs thereof.

18. The pharmaceutical composition of claim 14, wherein the solid dosage form is a sublingual tablet or a buccal troche.

19. The pharmaceutical composition of claim 14, wherein the liquid dosage form is selected from the group consisting of: sublingual solution, emulsion, suspension, and liquid spray.

20. The pharmaceutical composition of claim 14, wherein the semi-solid dosage form is selected from the group consisting of: gel, gel-forming ointment, and gel-forming paste.
